(19) United States
(12) Patent Application Publication
(10) Pub. No.: US 2021/0030006 A1
(43) Pub. Date: Feb. 4, 2021

(54) APPLICATION OF A NANO RNAi PREPARATION IN PVY PREVENTION AND CONTROL

(71) Applicants: Tobacco Research Institute of Chinese Academy of Agricultural Sciences, Qingdao City (CN); Shandong Peanut Research Institute, Qingdao City (CN); Zunyi Branch of Guizhou Tobacco Company, Zunyi City (CN)

(72) Inventors: Jinguang YANG, Qingdao City (CN); Manlin XU, Qingdao City (CN); Minghong LIU, Zunyi City (CN); Xiaoyan WANG, Zunyi City (CN); Yucheng CHI, Qingdao City (CN); Xia ZHANG, Qingdao City (CN); Fenglong WANG, Qingdao City (CN); Ying LI, Qingdao City (CN); Jie WANG, Qingdao City (CN); Lili SHEN, Qingdao City (CN); Jing YU, Qingdao City (CN); Zhiqing GUO, Qingdao City (CN)

(73) Assignees: Tobacco Research Institute of Chinese Academy of Agricultural Sciences, Qingdao City (CN); Shandong Peanut Research Institute, Qingdao City (CN); Zunyi Branch of Guizhou Tobacco Company, Zunyi City (CN)

(21) Appl. No.: 16/921,955

(22) Filed: Jul. 7, 2020

(30) Foreign Application Priority Data
Jul. 31, 2019 (CN) 201910702476.2

(51) Int. Cl. A01N 57/16; C12N 15/113

(52) U.S. Cl. CPC A01N 57/16; C12N 2310/14; C12N 15/1131

(57) ABSTRACT

The invention provides an application of a nano RNAi preparation in PVY prevention and control, belongs to the field of genetic engineering and application thereof, and can solve the problem of the nano RNAi preparation in PVY virus prevention and control. The RNAi preparation is prepared from dsRNA and chitosan nano materials, wherein the dsRNA is prepared from three gene capsid proteins CP, auxiliary components-protease HC-Pro and genome connexin VPg, and the dsRNA plays a key role in replication, proliferation and movement of PVY virus in plants. The nano RNAi preparation provided by the invention is used for virus prevention and control of PVY, so that the stability of dsRNA is stronger, the effect is longer, a good virus prevention and control effect can be achieved, and the nano RNAi preparation has a good application prospect in the field of PVY virus prevention and control.

### TECHNICAL FIELD

The invention belongs to the technical field of genetic engineering, and particularly relates to a novel application of a nano RNAi preparation in PVY prevention and control.

### BACKGROUND ART

With a wide range of hosts, potato virus Y (PVY) is a worldwide virus, which can cause plant vein necrosis and cause significant economic losses, but effective control measures is still lack for the virus.

In recent years, researches have reported that the problems of poor self-stability, easy degradation and the like of dsRNA are solved by combining a nano material with the dsRNA, and good prevention and control effects on certain plant viruses are achieved. As the Chinese patent document CN 109169702 A discloses the application of a nano RNAi preparation in TMV virus prevention and control, the prior art has multiple advantages of environmental friendliness, long lasting time, no harm to crops and the like, and can effectively control TMV virus. Although the nano RNAi preparation has a good effect in TMV virus prevention and control, little research is done on the application of the preparation in PVY virus prevention and control, so that the idea of the prior art can be effectively utilized, the preparation is applied to PVY virus prevention and control, and great significance is provided for large-scale prevention and control of the virus.

### SUMMARY OF THE INVENTION

The invention aims to provide an application of a nano RNAi preparation in PVY prevention and control, the preparation has the advantages of strong stability, difficulty in degradation, promotion of shuttle and transfer of dsRNA in plants to increase drug effect and the like, and PVY can be effectively prevented and controlled.

In order to achieve the above object, the invention adopts the following specific technical scheme: the invention provides an application of a nano RNAi preparation in PVY prevention and control, characterized in that the nano RNAi preparation is prepared from dsRNA and chitosan nano materials, and the dsRNA is prepared from three protein genes including capsid protein CP, auxiliary components-protease HC-Pro and genome connexin VPg, which play a key role in replication, proliferation and movement of PVY in plants.

Further, the preparation method of the dsRNA comprises following steps of:

selecting plant leaves infected by PVY, extracting total RNA of the plant leaves, and obtaining cDNA of PVY through a reverse transcription process;

designing amplification primers of CP, HC-Pro and VPg sequences by taking cDNA of the PVY as a template, and carrying out PCR amplification to obtain amplification products of the three genes;

synthesizing dsRNA in vitro by taking the amplification product as a template.

Further, the concentration of the dsRNA is 0.8-1.5 μg/μl.

Further, the method for extracting total RNA is Trizol extraction or to obtain through extraction according to the instructions of a total RNA extraction kit.

Further, the forward and reverse primer sequences of the CP gene are the DNA sequences shown in SEQ ID NOs. 1-2, the forward and reverse primer sequences of the HC-Pro gene are the DNA sequences shown in SEQ ID NOs. 3-4, and the forward and reverse primer sequences of the VPg gene are the DNA sequences shown in SEQ ID NOs. 5-6.

Further, the preparation method of the chitosan nano material is dissolving chitosan in 1% glacial acetic acid to prepare a chitosan nano material; the final concentration of the chitosan nano material solution is 1.5-2.5 μg/μl.

Further, the nano RNAi preparation is prepared by slowly adding dsRNA liquid to the chitosan nano material solution and uniformly mixing.

Further, the mass ratio of the chitosan to the dsRNA is (10-30): 1.

Further, the use method of the nano RNAi preparation is uniformly spreading or spraying a finished solution on plant leaves.

Further, the nano RNAi preparation can be applied to tobacco, tomato, pepper or potato.

Compared with the prior art, the advantages and positive effects of the invention are as follows:

(1) the invention provides an application of a nano RNAi preparation in PVY prevention and control, three gene capsid proteins CP, auxiliary components-protease HC-Pro and genome connexin VPg of PVY are used as templates to synthesize corresponding dsRNA in vitro, the stability of the dsRNA is improved through combination with a chitosan nano material, and RNAi in plants can be caused more stably;

(2) the preparation provided by the invention not only can increase the stability of the dsRNA, but also can promote the transfer and shuttle of the dsRNA in a plant, thereby achieving the purpose of increasing the drug effect;

(3) the preparation provided by the invention has the advantages that the concentration of dsRNA is 0.8-1.5 μg/μl, the concentration of the chitosan nano material solution is 1.5-2.5 μg/μl, and the mixing effect of the chitosan nano material solution and the dsRNA is good when the mixing ratio of the chitosan nano material solution and the dsRNA is (10-30: 1);

(4) the nano RNAi preparation provided by the invention can be applied to PVY prevention and control by spraying or fertilizing;

(5) the nano RNAi preparation provided by the invention can be used as a biological growth promoter and applied to related production in agriculture.

### BRIEF DESCRIPTION OF THE DRAWINGS

FIG. 1 is a image showing the result of agarose gel electrophoresis for extracting PVY RNA provided in Example 1 of the present invention, wherein, lanes from left to right are Marker, PVY RNA1, PVY RNA2, PVY RNA3, respectively;

FIG. 2 is a image of agarose gel electrophoresis results of in vitro transcriptionally synthesized dsRNA provided in Example 1 of the present invention, wherein, lanes from left to right are Marker, control, CP dsRNA, HC-Pro dsRNA and VPg dsRNA, respectively;

FIG. 3 is an agarose gel electrophoresis image of dsRNA provided in Example 1 of the present invention fused with chitosan in different ratios, wherein, lanes from left to right are Marker, CP dsRNA: chitosan 1:2, 1:5, 1:10, 1:20, 1:30; Marker, HC-Pro dsRNA: chitosan 1:2, 1:5, 1:10, 1:20, 1:30; Marker, VPg dsRNA: chitosan 1:2, 1:5, 1:10, 1:20, 1:30, respectively

FIG. 4 shows phenotypic results of tobacco leaves inoculated with PVY and dsRNA-chitosan according to Example 2 of the present invention, wherein 4-1, 4-2, 4-3, 4-4, 4-5 are respectively CP dsRNA+PVY tobacco susceptibility, HC-Pro dsRNA+PVY tobacco susceptibility, VPg dsRNA+PVY tobacco susceptibility, PVY tobacco susceptibility and negative control group infection.

### DETAILED DESCRIPTION OF THE INVENTION

Hereinafter, the technical scheme of the present invention will be clearly and completely described. It is to be understood that the described embodiments are only a few, but not all, embodiments of the invention. Based on the embodiments of the present invention, all other embodiments obtained by a person of ordinary skill in the art without involving any inventive effort are within the scope of the present invention.

The embodiment of the invention provides an application of the nano RNAi preparation in PVY prevention and treatment, the nano RNAi preparation is prepared from dsRNA and chitosan nano materials, and the dsRNA is prepared from three protein genes including capsid protein CP, auxiliary components-protease HC-Pro and genome connexin VPg, which play a key role in replication, proliferation and movement of PVY in plants.

dsRNA is prepared from three gene proteins CP, an auxiliary component-protease HC-Pro and a genome connexin VPg. The reason why the three genes are selected is that as a capsid protein of a virus, the CP gene wraps the genome single-stranded RNA of the virus, and meanwhile, has important influences on aspects of cell replication, proliferation and movement, aphid transmission, genome amplification and the like; hC-Pro plays an important role in genome amplification, self-interaction, long-distance transportation between cells and virus seed transmission; the VPg gene also plays an important role in the long-distance transportation and genome amplification process, so the three genes are very important in the replication, proliferation and movement of the virus, and the three important functional genes are silenced specifically through the RNAi technology, which is of great significance for the prevention and control of the PVY virus.

In a preferred embodiment, the method for preparing the dsRNA comprises the following steps of:

S1: selecting plant leaves infected by PVY, extracting total RNA of the plant leaves, and obtaining cDNA of PVY through a reverse transcription process;

S2: designing amplification primers of C-Pro, CP and VPg sequences by taking cDNA of PVY as a template, and carrying out PCR amplification to obtain amplification products of the three genes;

S3: synthesizing dsRNA in vitro by taking the amplification product as a template.

In a preferred embodiment, the concentration of dsRNA is 0.8-1.5 μg/μl.

The concentration of dsRNA was selected as 0.8, 0.9, 1.0, 1.1, 1.3, 1.5 μg/μl, etc.

In a preferred embodiment, the method for extracting total RNA is Trizol extraction or extraction according to the instructions of a total RNA extraction kit.

In a preferred embodiment, the forward and reverse primer sequences of the CP gene are the DNA sequences shown in SEQ ID NOs. 1-2, the forward and reverse primer sequences of the HC-Pro gene are the DNA sequences shown in SEQ ID NOs. 3-4, and the forward and reverse primer sequences of the VPg gene are the DNA sequences shown in SEQ ID NOs. 5-6.

In a preferred embodiment, the preparation method of the chitosan nano material is dissolving chitosan in 1% glacial acetic acid to prepare a chitosan nano material; the final concentration of the chitosan nano material solution is 1.5-2.5 μg/μ1.

The final concentration of chitosan nano material solution can be selected as 1.8, 1.9, 2.0, 2.2 or 2.5 μg/μl.

The invention also provides a preparation method of the nano RNAi preparation, which is prepared by slowly adding dsRNA liquid into the chitosan nano material solution and uniformly mixing.

In a preferred embodiment, the mass ratio of chitosan to dsRNA is (10-30): 1.

Wherein, if the mass ratio is too low, many dsRNA will not bind to chitosan, so that the prevention and control effect of PVY virus is finally influenced, and if the ratio is too high, many chitosan nano materials are not attached with dsRNA, resulting in unnecessary waste.

In a preferred embodiment, the use method of the nano RNAi preparation is uniformly spreading or spraying a finished solution on plant leaves.

Wherein, the carborundum is required to be added into the finished product solution during use, in order to make the leaves produce wounds, promote PVY virus infection, and ensure the success rate of virus inoculation, so as to better understand the prevention and control effect of chitosan dsRNA.

In a preferred embodiment, the nano RNAi preparation is applied to tobacco, tomato, pepper or potato.

In order to more clearly describe the nano RNAi preparation for PVY prevention and control and the preparation method thereof provided by the embodiment of the present invention, a description will be given below with reference to specific examples.

### EXAMPLE 1

A preparation method of the nano RNAi preparation for PVY prevention and control, comprises the following steps of:

(S1): the total RNA extraction kit is used for extracting the total RNA of the plant leaves, and the specific steps are as follows:

S1-1: weighing 50-100 mg of plant leaves and placing into a 1.5 ml RNA-Free centrifuge tube, rapidly grounding into powder in liquid nitrogen by using a grinding rod, and adding 500 μl SL added with δ-mercaptoethanol and uniformly vortexing. 12000 rpm, centrifuging for 2 min;

S1-2: transferring the supernatant to a filtration column CS, placing the filtration column CS in a collection tube, centrifuging at 12000 rpm for 2 min, and carefully sucking the supernatant in the collection tube to a new 1.5 ml RNA-Free centrifuge tube under the condition that a suction head avoids sucking bottom precipitate;

S1-3: adding 0.4 times the supernatant volume of anhydrous ethanol to the centrifuge tube. Uniformly mixing back and forth, completely transferring the solution into an adsorption column CR3, centrifuging at 12000 rpm for 30 s, pouring off waste liquid in a collection tube, and putting the adsorption column CR3 back into the collection tube;

S1-4: adding 350 μl of deproteinized liquid RW1 into an adsorption column CR3, centrifuging at 12000 rpm for 30-60 s, discarding waste liquid, and putting the adsorption column back into the collection tube;

S1-5: preparing DNasel solution: adding 70 μl RDD solution into 10 μl DNasel, and slowly and uniformly mixing;

S1-6: adding 80 μl DNasel solution into the adsorption column CR3, and standing for 15 min at room temperature;

S1-7: adding 350 μl of deproteinized liquid RW1 into CR3, centrifuging at 12000 rpm for 30-60 s, discarding waste liquid, and putting the adsorption column back into the collection tube;

S1-8: adding 500 μl of rinsing liquid RW (adding anhydrous ethanol) into the adsorption column CR3, standing for 2 minutes, centrifuging at 12000 rpm for 30-60 s, pouring off waste liquid, putting the adsorption column CR3 back into the collection tube, and removing impurities such as pigments and the like. Putting the adsorption column into a new collection tube, centrifuging at 12000 rpm for 2 min, standing the adsorption column CR3 at room temperature after centrifuging, ventilating and drying;

S1-9: putting the adsorption column CR3 in a RNase-Free centrifuge tube, adding 30-50 μl RNase-Free ddH2O, leaving at room temperature for 2 min, centrifuging at 12000 rpm for 2 min at 4° C.

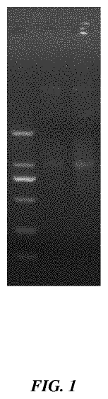

Finally, detecting the extracted RNA by 1.0% agarose gel electrophoresis, detection results as shown in FIG. 1, and then storing the extracted RNA in −80° C. ultra-low temperature refrigerator.

(S2): synthesizing PVY genome cDNA, a reverse transcription process is carried out by utilizing a FastKing RT Kit (With gDNase) Fast King cDNA first-strand synthesis kit from TIANGEN, and the specific operations are as follows:

S2-1: thawing template RNA on ice, thawing 5× g DNA Buffer, FQ-RY Primer Mix, 10× King RT Buffer and RNase-Free ddH2O at room temperature, then placing on ice, and finishing next few steps on ice;

S2-2: uniformly mixing 2 μl 5× g DNA Buffer and 8 μl RNA and centrifuging, incubating at 42° C. for 3 min, putting on ice after finishing;

S2-3: preparing a reverse transcription system mixed liquid system, the specific system is as follows: 10× King RT Buffer 2 μl; Fasting RT Enzyme Mix 1μl; FQ-RY Primer Mix 2 μl; RNase-Free ddH2O 5 μl;

S2-4: adding the solution generated in the reverse transcription reaction into the mixed solution prepared in the step (3), and uniformly mixing;

S2-5: incubating for 15 min at 42° C.;

S2-6: incubating for 3 min at 95° C., and storing the product in −80° C. ultra-low temperature refrigerator.

(S3): gene amplification of CP, HC-Pro and VPg

S3-1: designing primers of three genes according to sequence information, the primer sequences are shown in Table 1:

TABLE 1
Gene amplification primers of CP, HC-Pro
and VPg and corresponding sequences

| Gene | Primers | Sequence (5′-3′) |
|---|---|---|
| CP | PVYCP-F | 5′-ATTCTCTAGAAGCTTAATACGACTCAC |
| | (SEQ ID | TATAGGGATGACACAATTGATGCAGTAGAA |
| | NO. 1) | |
| | PVYCP-R | 5′-ATTCTCTAGAAGCTTAATACGACTCAC |
| | (SEQ ID | TATAGGGAGAGCATGCATACTTGGAGAGACA |
| | NO. 2) | |
| HC- | PVYHC-F | 5′-ATTCTCTAGAAGCTTAATACGACTCAC |
| Pro | (SEQ ID | TATAGGGATGGATTCAATGGTTCAGTTCTC |
| | NO. 3) | |
| | PVYHC-R | 5′-ATTCTCTAGAAGCTTAATACGACTCAC |
| | (SEQ ID | TATAGGGTTCCAATTTGCTTTGGCAGATAG |
| | NO. 4) | |
| VPg | PVYVPg-F | 5′-ATTCTCTAGAAGCTTAATACGACTCAC |
| | (SEQ ID | TATAGGGAATCCAAGCCTTGAAGTTTCGCC |
| | NO. 5) | |
| | PVYVPg-R | 5′-ATTCTCTAGAAGCTTAATACGACTCAC |
| | (SEQ ID | TATAGGGTTCATGCTCCACCTCCTGTGCTG |
| | NO. 6) | |

S3-2: performing PCR amplification reaction on the PVY genome by using the primers shown in Table 1 to obtain amplification products of three genes of CP, HC-Pro and VPg;

The PCR reaction system specifically comprises: taq mix 12.5 μl; Genomic DNA(PVY) 1 μl; Primer F 0.5 μl; Primer R 0.5 μl; H2O 11 μl;

PCR amplification conditions are as follows: pre-denaturation for 5 min at 95° C.; denaturation for 30 s at 95° C., Tm annealing for 30 s, extension for 1 min at 72° C., a total of 30 cycles; final extension for 10 min at 72° C., at last insulating at 12° C.

(S4): preparing dsRNA of three genes through in-vitro transcription, and carring out in-vitro transcription preparation according to instruction of a T7 RiboMAX™ Express RNAi System kit, and the specific operations are as follows:

S4-1: preparation of dsRNA: preparing a reaction system by taking 8 μl PCR amplification products, wherein the DNA in the linear PCR products can generate a single-strand transcription template strand, and taking the single-strand transcription template strand as an experimental group; while pGEM® Express Positive Control Template could not generate complete template strand, so it could not generate dsRNA, which was used as control group;

Preparation of a synthetic system of dsRNA in an experimental group: RiboMAX Express™ T7 2× buffer* 10 μl; linear DNA template 8 μl; Nuclease-Free Water 7 μl; Enzyme Mix, T7 Express 2 μl;

Preparation of a synthetic system of dsRNA in a control group: RiboMAX Express™ T7 2× buffer* 10 μl; pGEM® Express Positive Control Template 1 μl; Nuclease-Free Water 7 μl; Enzyme Mix, T7 Express 2 μl;

The system was prepared on an ice box, mixed gently and uniformly after the system was prepared, and incubating in a PCR instrument at a constant temperature of 37° C. for 3 h.

S4-2: removing the DNA template, annealing the dsRNA, and removing the ssRNA: Incubating the product of the previous step for 10 min in a PCR instrument at 70° C. and then slowly cooling to room temperature to anneal the dsRNA by this procedure. Diluting RNase Solution to 1: 200 and adding 1 μl RNase Solution to 199 μl deionized water. Taking 1 μl diluted RNase solution and 1 μl RQ1 RNase-Free DNase, and incubating in a PCR instrument for 30 min at 37° C., thereby removing residual single-stranded RNA and double-stranded DNA templates;

S4-3: purifying dsRNA: adding 2 μl 3 M Sodium Acetate (pH 5.2) and 50 μl 95% alcohol, mixing uniformly and placing on ice for 5 min, centrifuging at 12000 rpm for 10 min after turbidity, observing a white precipitate at the bottom, extracting and discarding the supernatant, washing the bottom precipitate with 0.5 ml pre-cooled 70% alcohol. Drying in air at room temperature for 15 min, after alcohol volatilization, adding 40-100 μl deionized water to obtain dsRNA;

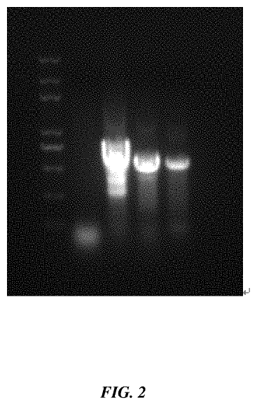

S4-4: detection of dsRNA: by agarose gel electrophoresis, adding dsRNA into a loading buffer to observe whether the bands are single and bright, and the electrophoresis detection results are shown in FIG. 2; and determining the concentration of dsRNA with a microspectrophotometer.

(S5) binding the chitosan nano material with dsRNA, the specific operation steps are as follows:

S5-1: dissolving chitosan in 1% glacial acetic acid to prepare a chitosan solution A with a final concentration of 2 μg/μl;

S5-2: slowly adding PVY dsRNA solutions with different concentrations into a chitosan solution A, respectively setting five mixing ratios of the three dsRNA of HC-Pro, CP and VPg, putting into a piped with sRNA: glacial acetic acid chitosan solution is 1:2, 1:5, 1:10, 1:20 and 1:30, and marking;

S5-3: mixing a chitosan solution containing dsRNA and a 1% SDS solution in a ratio of 6:3.5, wherein the former is slowly added into the latter, the addition sequence cannot be changed, and shaking for 10 min;

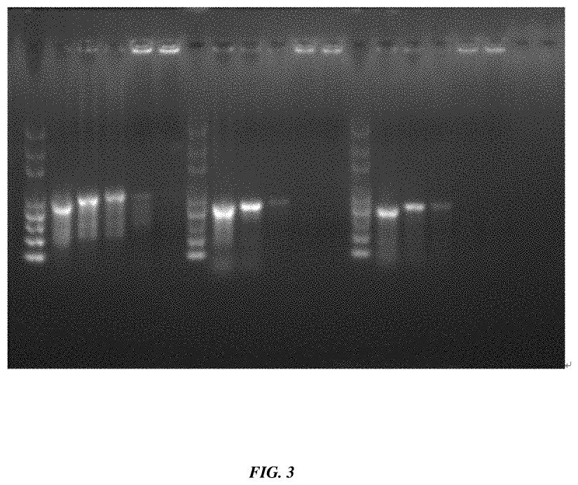

S5-4: 5 μl mixed solution was added to 1 μl 6× DNA loading buffer, and observing the results after agarose gel electrophoresis. If no bands were present and bright bands appeared around the wells, it was confirmed that the chitosan dsRNA was stable and successfully bound, and the results of fusion of dsRNA with different chitosans are shown in FIG. 3.

### EXAMPLE 2

The application of the nano RNAi preparation for PVY prevention and control in PVY virus prevention and control of embodiment 1, the specific application method is as follows:

S1: planting and transplanting laboratory tobacco:

Taking 15 disposable plastic cups, and burning the glass rod by the alcohol lamp to stamp holes at the bottom of the plastic cups, making it easy for soil absorb water. Mixing the nutrient soil and vermiculite in a ratio of 1:1 to ensure sufficient nutrition and strong water absorption capacity. Tobacco is firstly planted in the flowerpot, and is transplanted into a plastic cup after the plant height is suitable. In the transplanting process, the tobacco root is ensured to be complete, sufficient water supply is ensured, and the constant-temperature greenhouse illuminates for 18 h every day. Waiting for 2-3 weeks;

S2: inoculation of dsRNA with PVY:

grounding fluorescence-labeled GFP-PVY in liquid nitrogen, then adding 10 ml 1× buffer solution, adding a small amount of carborundum (generating wound), and coating 0.1 ml GFP-PVY on tobacco leaves gently and uniformly in one direction. In this experiment, PVY and dsRNA were inoculated at the same time, chitosan dsRNA preparation was applied to tobacco leaves by smearing or spraying, each treatment was repeated 3 times, only 3 pots of tobacco inoculated with GFP-PVY are in positive control group, 3 pots of tobacco sprayed with the same amount of chitosan solution are in negative control group.

S3: extraction of the whole protein of the plant, the specific steps are as follow:

The leaves inoculated with PVY and dsRNA-chitosan were used to extract protein by plant protein extraction kit for Western Blot.

(1) Grinding 150 mg plant leaves in liquid nitrogen;

(2) Adding 1 ml lysis solution, treating at 4° C. for 20 min, and shaking every 5 min;

(3) Centrifuge in a 4° C. high speed refrigerated centrifuge at 14000 rpm for 30 min;

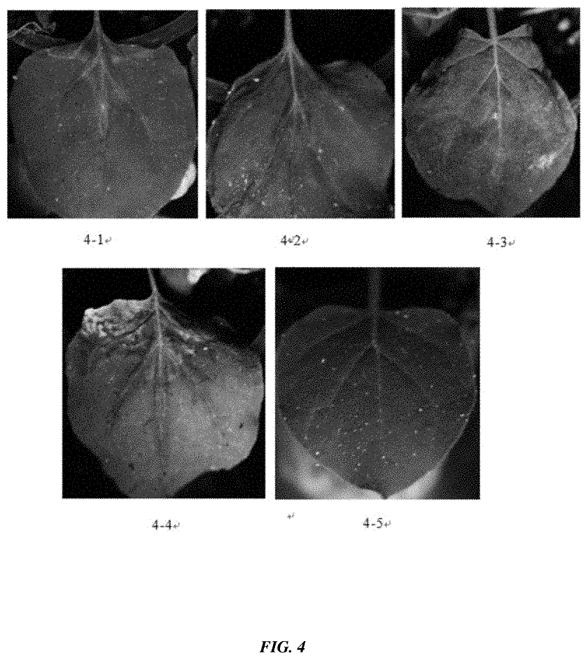

(4) Adsorbing supernatants into new centrifuge tubes, storing at −80° C., and phenotypes of tobacco leaf in each group are shown in FIG. 4.

The results of Western Blot showed that the expression of CP protein of PVY virus decreased significantly after three chitosan nano-preparations of CP-dsRNA, HC-dsRNA and VPg-dsRNA, indicating that three chitosan nano-preparations of CP-dsRNA, HC-dsRNA and VPg-dsRNA inhibited the replication and infection of PVY.

What is claimed is:

1. An application of a nano RNAi preparation in PVY prevention and control, characterized in that the nano RNAi preparation is prepared from dsRNA and chitosan nano materials, and the dsRNA is prepared from three protein genes including capsid protein CP, auxiliary components-protease HC-Pro and genome connexin VPg, which play a key role in replication, proliferation and movement of PVY in plants.

2. The application according to claim 1, characterized in that the preparation method of the dsRNA comprises following steps of:
selecting plant leaves infected by PVY, extracting total RNA of the plant leaves, and obtaining cDNA of PVY through a reverse transcription process;
designing amplification primers of CP, HC-Pro and VPg sequences by taking cDNA of the PVY as a template, and carrying out PCR amplification to obtain amplification products of the three genes;
synthesizing dsRNA in vitro by taking the amplification product as a template.

3. The application according to claim 2, characterized in that the concentration of the dsRNA is 0.8-1.5 μg/μl.

4. The application according to claim 2, characterized in that the method for extracting total RNA is Trizol extraction or to obtain through extraction according to the instructions of a total RNA extraction kit.

5. The application according to claim 2, characterized in that the forward and reverse primer sequences of the CP gene are the DNA sequences shown in SEQ ID NOs. 1-2, the forward and reverse primer sequences of the HC-Pro gene are the DNA sequences shown in SEQ ID NOs. 3-4, and the forward and reverse primer sequences of the VPg gene are the DNA sequences shown in SEQ ID NOs. 5-6.

6. The application according to claim 1, characterized in that the preparation method of the chitosan nano material is dissolving chitosan in 1% glacial acetic acid to prepare a chitosan nano material; the final concentration of the chitosan nano material solution is 1.5-2.5 μg/μl.

7. The application according to claim 1, characterized in that the nano RNAi preparation is prepared by slowly adding dsRNA liquid to the chitosan nano material solution and uniformly mixing.

8. The application according to claim 7, characterized in that the mass ratio of the chitosan to the dsRNA is (10-30): 1.

9. The application according to claim 1, characterized in that the use method of the nano RNAi preparation is uniformly spreading or spraying a finished solution on plant leaves.

10. The application according to claim 9, characterized in that the nano RNAi preparation can be applied to tobacco, tomato, pepper or potato.


SEQUENCE LISTING (9 sequences)

SEQ ID NO: 1 - DNA, 57 bp, Potato virus Y strain - NCBI - HM590405.1
attctctagaagcttaatacgactcactatagggatgacacaattgatgcagtagaa

SEQ ID NO: 2 - DNA, 58 bp, Potato virus Y strain - NCBI - HM590405.1
attctctagaagcttaatacgactcactatagggagagcatgcatacttggagagaca

SEQ ID NO: 3 - DNA, 57 bp, Potato virus Y strain - NCBI - HM590405.1
attctctagaagcttaatacgactcactatagggatggattcaatggttcagttctc

SEQ ID NO: 4 - DNA, 57 bp, Potato virus Y strain - NCBI - HM590405.1
attctctagaagcttaatacgactcactatagggttccaatttgctttggcagatag

SEQ ID NO: 5 - DNA, 57 bp, Potato virus Y strain - NCBI - HM590405.1
attctctagaagcttaatacgactcactatagggaatccaagccttgaagtttcgcc

SEQ ID NO: 6 - DNA, 57 bp, Potato virus Y strain - NCBI - HM590405.1
attctctagaagcttaatacgactcactatagggttcatgctccacctcctgtgctg

SEQ ID NO: 7 - DNA, 807 bp, Artificial
  Synthesized
atggcaaatgacacaattgatgcagtagaaagcaacaagaaagaatcaaaaccagagcaaggcagcatccagtcaaactcgaacaaaggaatagataaggatgtgaatgctggtacgtccggaacacatactgtgccgagaatcaaggctatcacgtccaaaatgagaatgcccaaaagcaagggagcaaccgtgctaaatttagaacacttgcttgagtacgctccacaacaaattgatatttcaaatactcgggcaactcaatcacagtttgatacgtggtatgaggcagtgcggatggcatacgacataggagaaactgagatgccaactgtgatgaatgggcttatggtttggtgcattgaaaatggaacctcgccaaatgtcaacggagtttgggttatgatggatgggaatgaacaagttgagtacccgttgaaaccaatcgttgagaatgcaaaaccaacccttaggcaaatcatggcacatttctcagatgttgcagaagcgtatatagaaatgcgcaacaaaaaggaaccatatatgccacgatatggtttaattcgaaatctgcgggatgtgggtttagcgcgttatgcctttgacttttatgaggtcacatcacgaacaccagtgagggctagggaagcgcacattcaaatgaaggccgcagcattgaaatcagcccaacctcgacttttcgggttggacggtggcatcagtacacaagaggagaacacagagaggcacaccaccgaggatgtctctccaagtatgcatgctctacttggagtcaagaacatgtga

SEQ ID NO: 8 - DNA, 1395 bp, Potato virus Y strain - NCBI - HM590405.1
ggggttatggattcaatggttcagttctcaagcgctgaaagcttttggaagggattggacggcaattgggcacaaatgagatatcctacagatcatacatgtgtggcaggtctaccagttgaagactgtggcagagttgcagcgataatgacacacagtattttaccgtgctataagataacctgccctacctgtgcccaacagtatgccaacttgccagccagtgacttacttaagatattacacaagcacgcaagtgatggtttaaatcgattaggggcagacaaagatcgctttgtgcatgtcaaaaagttcttaacaatcttagagcacttaactgaaccggttgatctgagtctagaaattttcaatgaagtgttcaagtctataggggagaagcaacaatcacctttcaaaaacctgaatattctgaataatttctttttgaaaggaaaggaaaatacagctcgtgaatggcaggtggctcaattaagcttacttgaattggcaagattccaaaagaacagaacggataatatcaagaaaggagacatttcgttctttaggaataaactatctgccaaagcaaattggaacttgtatctgtcatgtgataaccagctggataagaatgcaaacttcctgtggggacagagggaatatcatgctaagcgatttttctcgaattatttcgaggaaattgatccagcgaagggctattcagcatacgaaaatcgtttgcatccgaatgggacaagaaaacttgcaattggaaacctaattgtaccacttgatctggctgagtttaggcggaagatgaaaggtgattataaaagacagccaggggtgagtaagaagtgcacgagctcgaaggatggaaactacgtgtatccctgttgttgcactacacttgatgatggctcagctgttgaatcaacattttacccgccaactaagaagcacctcgtaataggtaatagtggcgaccaaaagtatgttgacttaccaaaagggaattctgagatgttatatattgccaggcaaggcttctgttacattaacattttcctcgcgatgttgattaacattagtgaggaagatgcaaaggatttcactaagaaggttcgtgacatgtgtgtgccaaagcttggaacctggccaaccatgatggatctggctacaacttgtgctcaaatgaaaatattctaccctgatgttcatgatgcagaactgcctagaatactagtcgatcacgaaacacagacatgccatgtggttgactcgtttggctcacaaacaactgggtatcatattctaaaagcatccagcgtatctcaacttatcttgtttgcaaatgatgaattagaatctgatataaaacattatagagttggt

SEQ ID NO: 9 - DNA, 564 bp, Potato virus Y strain - NCBI - HM590405.1
gggaaaaataaatccaaaagaatccaagccttgaagtttcgccatgctcgtgacaaaagggctggctttgaaattgacaacaatgatgacacaatagaggaattcttcggatctgcatacaggaaaaagggaaaaggtaaaggtaccacagttggtatgggtaagtcaagcaggaggttcatcaacatgtatgggtttgatccaacagagtactcattcatccaattcgttgatccactcactggggcgcaaatagaagaaaatgtctatgctgacattagagatattcaagagagatttagtgaagtgcgaacgaaaatggttgagaatgatgacattgaaatgcaagccttgggtagtaacacgaccatacatgcatacttcaggaaagattggtctgacaaagctttgaagattgatttaatgccacataacccactcaaagtttgtgacaaaacaaatggcattgccaaatttcctgagagagagctcgaactaaggcagactgggccagctgtagaagtcgacgtgaaggacataccagcacaggaggtggagcatgaa